(19) United States
(12) Patent Application Publication
(10) Pub. No.: US 2023/0417741 A1
(43) Pub. Date: Dec. 28, 2023

(54) NANOPARTICLE-ENABLED ANALYSIS OF CELL-FREE NUCLEIC ACID IN COMPLEX BIOLOGICAL FLUIDS

(71) Applicant: THE UNIVERSITY OF MANCHESTER, Manchester (GB)

(72) Inventors: Marilena HADJIDEMETRIOU, Manchester (GB); Kostas KOSTARELOS, Manchester (GB); Lois GARDNER, Manchester (GB)

(21) Appl. No.: 18/041,141

(22) PCT Filed: Aug. 9, 2021
(86) PCT No.: PCT/GB2021/052055

§ 371 Date: Feb. 9, 2023

(30) Foreign Application Priority Data
Aug. 10, 2020 (GB) 2012433.5

(51) Int. Cl. G01N 33/53; G01N 33/543; G01N 33/574

(52) U.S. Cl. CPC G01N 33/5308; G01N 2800/56; G01N 33/57488; G01N 33/5432

(57) ABSTRACT

The invention relates to methods for capturing, quantifying and analyzing cell free nucleic acid (cfNA) in biofluid samples, such as blood and urine.

### FIELD OF THE INVENTION

The present invention relates to methods for capturing, quantifying and analyzing cell free nucleic acid (cfNA) in biofluid samples, such as blood and urine. Such method may be useful in detecting and quantifying disease specific biomarkers. In particular, the methods involve contacting nanoparticles with a biofluid from a subject, optionally in a diseased state, and subsequent analysis of the biomolecule corona formed on said nanoparticles. In addition, the present invention relates to methods for early disease detection and monitoring of disease progression in a subject by assessing the amount of disease-specific cfNA over time.

### INTRODUCTION

Research into cell free nucleic acid biomarker detection has been carried out but so far has failed to provide suitable methods to accurately identify/discover and detect disease-specific cfDNA. One particular problem is the extremely low concentration of cfDNA in biofluids. Furthermore, such methods are mainly used to detect already known disease-specific nucleic acid molecules (such as activating mutations associated with cancer).

Over the last decade, biomedical applications of nanoparticles (NPs) have been challenged due to the spontaneous adsorption of biomolecules onto their surface upon incubation with complex biofluids, known as the ‘protein’ or “biomolecule corona” (Hadjidemetriou and Kostarelos, Nat. Nanotechnol., 2017, 12, 288-290). The bio-nanotechnology field has since invested considerable resources investigating the corona composition in an attempt to prevent NP-protein interactions and consequently limit opsonisation-mediated clearance from blood and masking of surface ligands (Dai et al. Adv. Healthc. Mater., 2018, 7, 1700575; Chen et al. Nanoscale, 2019, 11, 8760-8775; Debayle et al. Biomaterials, 2019, 219, 119357; and Müller et al. Biomaterials, 2017, 115, 1-8). Protein corona formation is now a widely accepted phenomenon and has been documented for a wide range of NPs, including lipid-, metal-, polymer- and carbon-based nanomaterials, with their composition and surface chemistry altering the specific classes of proteins adsorbed (Docter et al. Nanomedicine, 2015, 10, 503-519).

WO2018/046542 discloses a proteomic biomarker discovery platform utilising a nanoparticle protein corona that enables a higher-definition, in-depth analysis of the blood proteome and the enrichment of low abundant disease-specific proteins.

Despite recent advances in analysing the blood-circulating genome, very little attention has been placed on the utilisation of the spontaneous interaction of NPs with nucleic acids upon incubation with biological fluids.

Surprisingly, the inventors have found that the biomolecule corona formed on nanoparticles after administration of nanoparticles to animal subjects or incubation of nanoparticles in a biofluid sample taken from a human subject in a healthy or in a diseased state results in interaction of the nanoparticles with cell free nucleic acid biomolecules as well as protein biomarkers. In one embodiment, the novel methods take advantage of the interaction of nanoparticles with nucleic acid biomolecules as a way to facilitate the detection of previously unknown disease-specific biomolecules.

### SUMMARY OF THE INVENTION

The inventors have found that cfNA exists in the biomolecule corona formed around NPs in human plasma, and at quantifiable levels. The inventors incubated clinically-used liposomes with plasma samples, retrieved the corona-coated liposomes, and subsequently quantified the total corona cfDNA content using two different real-time quantitative PCR (qPCR) assays. The data confirmed the presence of cfDNA molecules in the biomolecule corona. In addition, analysis of the liposome corona formed in plasma samples obtained from ovarian carcinoma patients revealed higher total cfDNA content compared to healthy controls, suggesting a disease-specific biomolecule corona.

According to a first aspect of the invention there is provided a method of capturing and analyzing cell free nucleic acid (cfNA) in a biofluid, wherein the method comprises:
(a) contacting a plurality of nanoparticles with a biofluid of a subject to allow a biomolecule corona to form on the surface of said nanoparticles;
(b) isolating the nanoparticles and surface-bound biomolecule corona; and
(c) analyzing the biomolecule corona for cfNA.

In particular embodiments, step (a) is performed in vivo by administering a plurality of nanoparticles to a subject or ex vivo using a biofluid sample that has been taken from the subject. In this embodiment, the subject is typically a human or a non-human animal, such as a mouse, rat or monkey.

The biofluid may be selected from blood, plasma, urine, saliva, lacrimal, cerebrospinal and ocular fluids, or any combination thereof. Suitably the biofluid is a blood or blood fraction sample, such as serum or plasma, and urine samples. Suitably, the blood or blood fraction sample is from circulating blood.

The method of the first aspect of the invention may be used to identify new biomarkers (for example, cell free nucleic acid biomarkers).

Suitably the method involves identification of a biomarker that provides a measurable indicator of some biological state or condition. This includes, but is not limited to, the discovery of unique disease-specific biomolecules (disease-specific mutations). It will be understood that in order to identify a potential disease-specific biomarker, comparison against a suitable non-diseased control reference can be required.

In one particular embodiment, the methods involve identifying panels of biomarkers (multiplexing), which can lead to increased sensitivity and specificity of detection.

The method may be used to identify new previously unknown biomarker, e.g. disease-associated biomarkers. In a particular embodiment, the unknown biomarker is a unique biomolecule, meaning that it is a biomolecule that would not have been detected if analysis was carried out directly on biofluid, such as plasma, isolated from the subject.

In a further particular embodiment, the methods facilitate the detection of previously unknown unique disease-specific biomolecules.

In yet a further particular embodiment, the methods allow identification or detection of a biomarker without the need for invasive tissue sampling, e.g. a biopsy.

The methods are applicable to a wide range of nanoparticles and allow the benefit of removal of unbound and highly abundant biomolecules to allow identification of low abundant biomarkers that would otherwise be undetected. In addition to identification of potential biomarkers, the methods can also be employed to monitor changes in biomarkers, for example in response to therapy and/or to assist in diagnosis.

The methods disclosed herein are applicable to any disease state in which identification/detection and/or monitoring of biomarkers would be beneficial. Furthermore, particular methods of the invention, which can be employed to distinguish between healthy and diseased states in a subject, are applicable to a wide range of diseases, including but not limited to, cancer and neurodegenerative diseases. In particular, the methods of the invention can be used to diagnose a disease, such as cancer, including in the early detection of a diseased state such as the presence of a cancer or pre-cancerous condition in a human subject.

According to a second aspect of the invention there is provided a method for detecting a disease state in a subject comprising:
(a) contacting a biofluid sample from the subject with a plurality of nanoparticles under conditions to allow a biomolecule corona to form on the surface of said nanoparticles;
(b) isolating the nanoparticles and surface-bound biomolecule corona; and
(c) analyzing the biomolecule corona for a disease-specific cfNA biomarker, which is determinative of the presence of a disease in said subject.

According to a third aspect of the invention there is provided a method for diagnosing cancer in a subject, comprising:
(a) contacting a biofluid sample from the subject with a plurality of nanoparticles under conditions to allow a biomolecule corona to form on the surface of said nanoparticles;
(b) isolating the nanoparticles and surface-bound biomolecule corona; and
(c) analyzing the biomolecule corona for cfNA;
   wherein an increase in total cfNA level relative to a reference or control amount is indicative of the presence of cancer.

The method can be used to monitor disease progression, for example to monitor the efficacy of a therapeutic intervention.

Suitably the disease is cancer. In a particular embodiment, the cancer is ovarian cancer.

According to a fourth aspect of the invention there is provided a method for monitoring disease progression in a subject, comprising:
(a) contacting a biofluid sample from the subject with a plurality of nanoparticles under conditions to allow a biomolecule corona to form on the surface of said nanoparticles;
(b) isolating the nanoparticles and surface-bound biomolecule corona; and
(c) analyzing the biomolecule corona for cfNA;
   wherein the degree of disease progression is determined based on the total cfNA level or cfNA level of a disease-specific biomarker relative to a reference amount.

Suitably, in any of the aspects of the invention, the cfNA is cell free ribonucleic acid (cfRNA) or cell free deoxyribonucleic acid (cfDNA).

In a particular embodiment, the disease is cancer.

Suitably, in any of the aspects of the invention, the biofluid may be selected from blood, plasma, urine, saliva, lacrimal, cerebrospinal and ocular fluids, or any combination thereof. Suitably, the biofluid is a blood or blood fraction sample, such as serum or plasma, and urine. Suitably, the blood or blood fraction sample is from circulating blood.

Any embodiment described herein can be applied to any aspect of the invention unless indicated otherwise or it is apparent to the person of skill in the art that such embodiment cannot apply.

### DESCRIPTION OF THE DRAWINGS

In order that the invention may be more clearly understood one or more embodiments thereof will now be described, by way of example only, in relation to an experimental study and with reference to the accompanying drawings, of which:

FIG. 1—Schematic representation of sample pre-processing and cfDNA quantification method pipelines. A) Schematic overview of human plasma and liposomal nanoparticle (NP) incubation and subsequent size-exclusion purification methodology. B) Method analysis pipeline for plasma processing (including cfDNA purification) and subsequent q-PCR quantification of cfDNA in NP corona samples and plasma control samples.

FIG. 2—Characterisation of cfDNA content in the healthy ex vivo biomolecule corona. A) cfDNA and liposomal lipid quantification across 15 chromatographic fractions. The purified cfDNA from a single healthy pooled plasma sample incubated with and without liposomal nanoparticles (NPs) was quantified by a highly-sensitive LINE-1 real-time PCR assay. NPs and cfDNA are expressed as percentage (%) of total recovered across chromatographic fractions. B) RNase P real-time cfDNA quantification of pooled ex vivo NP⁽⁺⁾ corona samples and NP⁽⁻⁾ controls (size-purified plasma). cfDNA was measured directly and in samples with additional cfDNA purification step. C) cfDNA concentrations in NP⁽⁺⁾ corona samples and NP ⁽⁻⁾ controls were confirmed using the LINE-1 real-time PCR assay. For graphs B and C cfDNA is expressed as percentage recovery (%) relative to QIAGEN's QIAamp® Circulating Nucleic Acid extraction kit (average of three replicates). All error bars represent mean and standard deviation. Groups were compared using a student t-test (p values<0.05 were considered significant).

FIG. 3—Assessing the accuracy of direct real-time PCR cfDNA quantification in ex vivo healthy and disease nanoparticle corona samples. A) RNase P real-time qPCR quantification of in pooled healthy liposomal corona samples and liposome⁽⁻⁾ plasma controls. B) Direct RNase P qPCR inhibition determined using 2-fold dilution of pooled NP corona samples. C-D) LINE-1 real-time qPCR quantification of cfDNA in late-stage serous ovarian cancer ex vivo biomolecule corona samples (n=8). Graph C represents cfDNA in NP corona samples and NP corona purified cfDNA, whereas graph D represents cfDNA in unpurified plasma (diluted 1:40) and purified plasma. All error bars represent mean and standard deviation. Groups were compared using a student t-test was performed (adjusted p values<0.05 were considered significant). E) Clinical details of eight late-stage ovarian cancer plasma samples included in graphs C and D.

FIG. 4—Reproducibility & linearity experiments of healthy plasma NP corona samples. A) Reproducibility data showing the percentage recovery (%) of QIAamp® purified NP corona cfDNA across liposome NP batches relative to QIAamp extracted plasma cfDNA (100%). B-C) Linearity data to investigate the effect of liposome concentration and plasma volume on cfDNA content in the liposome biomolecule corona. B) Graph highlighting the effect of plasma volume on cfDNA concentration (ng cfDNA/sample). Standard protocol 820 μL plasma: 180 μL liposomes. C) Graph showing the effect of liposome concentration on cfDNA concentration (ng cfDNA/sample). 12.5 mM liposomes represent standard protocol. All error bars represent mean and standard deviation. Three groups or more were compared using a one-way analyses of variance (ANOVA) test followed by the Tukey's multiple comparison test. Adjusted p values<0.05 were considered significant.

FIG. 5—Cell-free DNA (cfDNA) detection in the ex vivo ovarian cancer biomolecule corona. A) Normalised cfDNA concentration (ng/μM lipid) in corona-coated liposomes (ovarian cancer samples and age- and sex-matched healthy controls), measured using a highly-sensitive LINE-1 real-time PCR assay and robust inhibitor-resistant polymerase. B) The same data with ovarian cancer patients separated into early stage (1 & 2) and late-stage (3 & 4) cancers. All error bars represent mean and standard deviation. Three groups or more were compared using a one-way analyses of variance (ANOVA) test followed by the Tukey's multiple comparison test. For comparisons of two groups a student t-test was performed (adjusted p values<0.05 were considered significant).

FIG. 6—Histone proteins identified by LC-MS/MS in the biomolecule corona of healthy and ovarian cancer female plasma samples. A) LC-MS/MS normalised protein abundance of histones H2A, H2B and H4 in ovarian cancer corona samples and age-matched healthy corona controls. A one-way ANOVA was performed by the Progensis QI software with significance bars representing FDR-adjusted p values. B) Table summarising the relative abundance of proteins identified by LC-MS/MS associated with nucleosomes (DNA-histone complex) known to contain cfDNA. Max fold change between ovarian cancer corona samples and healthy corona controls is provided with FDR-adjusted p value from a one-way ANOVA in Progensis QI).

FIG. 7—Physiochemical characterisation of liposome nanoparticles (NPs). A) Graphs representing the size (diameter in nm) and zeta-potential distribution (mV) of PEG:HSPC:CHOL liposome batches 1-3. B) Table listing the mean average size (nm), polydispersity index (PDI) and zeta-potential (mV) of each liposome batch including standard deviations.

### DETAILED DESCRIPTION OF THE INVENTION

The practice of particular embodiments of the invention will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred embodiments of compositions, methods and materials are described herein.

### Definitions

The articles “a,” “an,” and “the” are used herein to refer to one or to more than one (i.e. to at least one) of the grammatical object of the article.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

The term “biomolecule” includes, but is not limited to, proteins, peptides, fatty acids, lipids, amino acids, amides, sugars and nucleic acids (such as for example different types of DNA or RNA).

As used herein, the term “disease-specific biomarker” refers to a biomarker which is associated with or indicative of a disease. Examples of certain cancer-specific biomarkers include: mutations in genes of KRAS, p53, EGFR or erbB2 for colorectal, esophageal, liver, and pancreatic cancer; mutations in BRCA1 and BRCA2 genes for breast and ovarian cancer; and, abnormal methylation of tumor suppressor genes p16, CDKN2B, and p14ARF for brain cancer.

As used herein, the term “high-throughput sequencing” is also referred to as “second-generation sequencing,” and the principles of high-throughput sequencing techniques are well known to those of skill in the art, and high-throughput sequencing is typically performed on microporous chips. High throughput sequencing techniques and the reagents and devices used therein are conventional in the art. Commercially available high throughput sequencing chips and reagents are readily available, for example, from Life Technologies Inc. To conduct high throughput sequencing the cfDNA captured in the corona may need a pre-treatment process such as amplification, end-repair, ligation, labeling and/or purification, etc. in order to construct a cfDNA library prior to high-throughput sequencing, and the techniques required for this are understood by those of skill in the art of high-throughput sequencing, and can be constructed, for example, using the NEBNext Fast DNA Fragmentation & Library Prep Set for Ion Torrent (Life Technologies Cat. No. 4474180) kit.

As used herein, the term “in vitro” means performed or taking place in a test tube, culture dish, or elsewhere outside a living organism. The term also includes ex vivo because the analysis takes place outside an organism.

As used herein, the term “isolated” means material that is substantially or essentially free from components that normally accompany it in its native state. In particular embodiments, the term “obtained” or “derived” is used synonymously with isolated.

A “target genetic locus” or “nucleic acid target region” refers to a region of interest within a nucleic acid sequence. In various embodiments, targeted genetic analyses are performed on the target genetic locus. In particular embodiments, the nucleic acid target region is a region of a gene that is associated with a particular genetic state, genetic condition, genetic diseases; genetic mosaicism, predicting response to drug treatment; diagnosing or monitoring a medical condition; microbiome profiling; pathogen screening; or organ transplant monitoring.

As used herein “targeted genetic analyses” refers to investigations of specific known genetic regions, including mutations, for example those that are known to be associated with a disease. Exemplary genetic regions include genes (e.g. any region of DNA encoding a functional product) or a part thereof, gene products (e.g., RNA and expression of genes). The genetic regions can include variations with the sequence or copy number. Exemplary variations include, but are not limited to, a single nucleotide polymorphism, a deletion, an insertion, an inversion, a genetic rearrangement, a copy number variation, or a combination thereof. The methods of the invention can be used to isolate cfNA that can then be subjected to any desired targeted genetic analysis.

As used herein, the terms “circulating NA,” “circulating cell-free NA” and “cell-free NA” are often used interchangeably and refer to nucleic acid that is extracellular DNA or RNA, DNA or RNA that has been extruded from cells, or DNA or RNA that has been released from lysed, necrotic or apoptotic cells.

A “subject,” “individual,” or “patient” as used herein, includes any animal that exhibits a symptom of a condition that can be detected or identified with compositions contemplated herein. Suitable subjects include laboratory animals (such as mouse, rat, rabbit, or guinea pig), farm animals (such as horses, cows, sheep, pigs), and domestic animals or pets (such as a cat or dog). In particular embodiments, the subject is a mammal. In certain embodiments, the subject is a non-human primate and, in a particular embodiment, the subject is a human.

### Methods of the Invention

According to a first aspect of the invention there is provided a method of identifying cell free nucleic acid (cfNA) in a biofluid, wherein the method comprises:
(a) contacting a plurality of nanoparticles with a biofluid of a subject to allow a biomolecule corona to form on the surface of said nanoparticles;
(b) isolating the nanoparticles and surface-bound biomolecule corona; and
(c) analyzing the biomolecule corona for cfNA.

Advantageously, the method according to the first aspect is used to identify cell free nucleic acid (cfNA) in a biofluid. It is to be understood that the term “identify” in this context relates to discovering cfNA which are new (i.e., previously not known and/or previously not associated with a particular disease or stage of disease that the subject from which the biofluid was taken has). In one embodiment, there is provided the method according to the first aspect wherein the method identifies cfNA in a biofluid from a subject in a diseased state wherein the biomarkers have previously not associated with a particular disease or stage of disease.

In particular embodiments, step (a) is performed in vivo by administering a plurality of nanoparticles to a subject or ex vivo or in vitro using a biofluid sample that has been taken from the subject.

In a particular embodiment, step (a) is performed in vivo by administering a plurality of nanoparticles to a human subject, a biofluid sample is then taken from the subject and analysed. In a particular embodiment, step (a) is performed in vivo by administering a plurality of nanoparticles to a non-human subject, a biofluid sample is then taken from the subject and analysed. Suitably, in step (b) the particles are isolated from the biofluid and purified to remove unbound and highly abundant biomolecules. In one embodiment the nanoparticles are administered to the subject by intravenous injection.

According to a variation of the first aspect of the invention there is provided a method of identifying cell free nucleic acid (cfNA) in a biofluid, wherein the method comprises:
(a) administering a plurality of nanoparticles to a subject to allow a biomolecule corona to form on the surface of said nanoparticles;
(b) isolating the nanoparticles and surface-bound biomolecule corona; and
(c) analyzing the biomolecule corona for cfNA.

In this approach, step (a) of the method involves administering a plurality of nanoparticles to a subject to allow a biomolecule corona to form on the surface of said nanoparticles. Suitably, the subject is human. Suitably, the subject is a non-human animal, such as a mouse, rat or monkey. Suitably, administration can be by any route that allows the biomolecule corona to form. Suitable routes of administration include but are not limited to intravenous, oral, intracerebral (including spinal), intraperitoneal and intra-occular. Conveniently, the route of administration is by intravenous injection. The biomolecule corona typically forms within less than 10 minutes from administration. Suitably, the subject is suffering from a disease (is in a diseased state).

A biofluid sample comprising some of the introduced nanoparticles is then extracted from the subject; for example, by taking a blood sample. In a particular embodiment, the nanoparticles are isolated from the biofluid sample prior to analysis. Any isolation technique that is capable of preserving the surface-bound biomolecule corona is suitable. Conveniently, the nanoparticles with surface-bound biomolecule corona are isolated from the biofluid and purified to remove unbound and biomolecules. Conveniently, the isolation is achieved by a method comprising size exclusion chromatography followed by ultrafiltration.

According to another variation of the first aspect of the invention there is provided a method of identifying cell free nucleic acid (cfNA) in a biofluid, wherein the method comprises:
(a) incubating a plurality of nanoparticles in a biofluid sample taken from a subject to allow a biomolecule corona to form on the surface of said nanoparticles;
(b) isolating the nanoparticles and surface-bound biomolecule corona; and
(c) analyzing the biomolecule corona for cfNA.

In this approach, the NP corona is formed ex vivo by incubating the plurality of nanoparticles in a biofluid sample to be analyzed. Suitably, such incubation can be carried out ex vivo or in vitro (herein the term in vitro includes ex vivo). Conveniently, this involves incubating at a suitable temperature, such as at about 37° C., for a suitable length of time. The biomolecule corona can form almost immediately, but typically the incubation is carried out for a period of 5-60 minutes, or more; such as 5, 10, 15, 20, 30, 40, 50, 60 or more minutes. Conveniently, the mixture can be subject to agitation, for example by way of an orbital shaker set at approximately 250 rpm to mimic in vivo conditions. Suitably, the biofluid sample from the subject to be analyzed has been previously taken and the sample extraction step is not part of the method.

Thus, according to a particular embodiment, the plurality of nanoparticles are incubated in the test biofluid sample ex vivo under conditions to allow a biomolecule corona to form on the surface of said nanoparticles.

In accordance with the first aspect of the invention, the corona may be digested prior to step c) in order to facilitate analysis.

In embodiments where the non-diseased control reference comprises a biomolecule corona obtained from a healthy subject, said corona may be digested prior to the equivalent steps of its own analysis.

The methods of the first aspect of the invention may also be useful for monitoring changes in the amount of the biomarkers, for example in response to therapy. Therefore, in some embodiments, the method may comprise an extra step, during or (preferably before step a) of administering a therapy to the subject, for example administering a drug molecule, such as for example, an anti-cancer compound. Suitable anti-cancer compounds include, but are not limited to, compounds with activity in cancers such as lung cancer, prostate cancer, melanoma or ovarian cancer. In some embodiments, the anti-cancer compound is doxorubicin.

The results obtained in step (c) can be compared to a non-diseased control reference which may comprise the results of corona analysis obtained from a healthy subject. The corona obtained from a healthy subject may be obtained by the same or similar method steps as steps a) and b) of the method, and may be analyzed by the same or similar method step as step c) of the method. The healthy subject may be a subject who does not have the type of disease (e.g. cancer) for which the likelihood thereof is being assessed, who does not have any form of disease and/or who does not have any serious illnesses or diseases (e.g. a subject who is generally considered, for example by doctors or other medical practitioners, to be healthy and/or substantially free from disease or illness or serious disease or illness).

A further step d) may comprise determination and/or calculation of relative or differential abundance between the corona and the non-diseased control reference (such as analysis results of a corona obtained by the same or similar method steps as steps a) to c) of the method, but wherein the subject is a healthy subject) with respect to the or each of the one or more biomarkers. Step c) and/or d) may comprise the use of a computer program or software tool. Step c) and/or d) may comprise analysis (such as computer or software analysis) of raw data obtained from analyses and/or measurements, for example raw data obtained from LC/MS of the or each corona. Step c) and/or d) may comprise a statistical comparison between the protein abundance of the one or more protein biomarkers in the corona

The corona may be digested prior to step c) and/or step d), in order to facilitate analysis. In embodiments where the non-diseased control reference comprises a biomolecule corona obtained from a healthy subject, said corona may be digested prior to the equivalent steps of its own analysis. Stroun et al. (Neoplastic characteristics of the DNA found in the plasma of cancer patients. Oncology. 46 (5): 318-322, 1989) described that certain characteristics of tumour DNA could be found in a patient's cfDNA. Subsequent publications have confirmed that tumour cells can release their DNA into the circulation. In 1996 Chen et al. (Nat. Med 2:1033-1035, 1996) and Nawroz et al. (Nat. Med 2:1035-1037, 1996) reported the presence of tumour-associated microsatellite alterations, such as loss of heterozygosity (LOH) and microsatellite shifts, in serum and plasma of cancer patients. Circulating free DNA is therefore a useful source material for cancer diagnosis and monitoring.

The inventors have found that analysis of the liposome corona formed in plasma samples obtained from ovarian carcinoma patients revealed higher total cfDNA content compared to healthy controls, suggesting a disease-specific biomolecule corona.

Thus, according to particular embodiments, the method can be used to diagnose or monitor a disease, such as cancer. Suitable cancers include ovarian, lung, melanoma, prostate and blood cancer, including leukemia, lymphoma and myeloma.

The method may be useful in the early detection of a diseased state such as the presence of a tumour in a human subject or for monitoring disease progression and/or response to treatment without the need for invasive tissue sampling, e.g. a biopsy.

According to a second aspect of the invention there is provided a method for detecting a disease state in a subject comprising:
(a) contacting a biofluid sample from the subject with a plurality of nanoparticles under conditions to allow a biomolecule corona to form on the surface of said nanoparticles;
(b) isolating the nanoparticles and surface-bound biomolecule corona; and
(c) analyzing the biomolecule corona for a disease-specific cfNA biomarker, which is determinative of the presence of a disease in said subject.

As with the first aspect of the invention, step (a) of this second aspect of the invention involve administering a plurality of nanoparticles to a subject to allow a biomolecule corona to form on the surface of said nanoparticles or incubating a plurality of nanoparticles in a biofluid sample taken from a subject to allow a biomolecule corona to form on the surface of said nanoparticles. Suitably, the subject is human. Whilst this approach can be conducted in humans, suitably, the subject is a non-human animal such as a mouse, rat or monkey.

In a further embodiment of the second aspect, step (a) comprises incubating a plurality of nanoparticles in a biofluid sample taken from a subject to allow a biomolecule corona to form on the surface of said nanoparticles. Suitably, such incubation can be carried out ex vivo or in vitro (herein the term in vitro includes ex vivo). In this approach, the NP corona is formed in vitro by incubating the plurality of nanoparticles in a biofluid sample to be analyzed. Conveniently, this involves incubating at a suitable temperature, such as at about 37° C., for a suitable length of time. The biomolecule corona can form almost immediately, but typically the incubation is carried out for a period of 5-60 minutes, or more; such as 5, 10, 15, 20, 30, 40, 50, 60 or more minutes. Conveniently, the mixture can be subject to agitation, for example by way of an orbital shaker set at approximately 250 rpm to mimic in vivo conditions. Suitably, the biofluid sample from the subject to be analyzed has been previously taken and the sample extraction step is not part of the method.

In step (b), any isolation technique that is capable of preserving the surface-bound biomolecule corona is suitable. Conveniently, the nanoparticles with surface-bound biomolecule corona are isolated from the biofluid and purified to remove unbound to allow identification of lower abundant biomarkers. Conveniently, the isolation is achieved by a method comprising size exclusion chromatography followed by ultrafiltration.

The invention is particularly suited for use in diagnosing cancer.

According to a third aspect of the invention there is provided a method for diagnosing cancer in a subject, comprising:
(a) contacting a biofluid sample of the subject with a plurality of nanoparticles under conditions to allow a biomolecule corona to form on the surface of said nanoparticles;
(b) isolating the nanoparticles and surface-bound biomolecule corona; and
(c) analyzing the biomolecule corona for cfNA;
wherein an increase in total cfNA level relative to a reference or control amount is indicative of the presence of cancer.

Suitably, the cancer is lung, ovarian, melanoma, prostate and blood cancer, including leukemia, lymphoma and myeloma. Suitably, the cancer is ovarian cancer. In one embodiment, there is provided a method for diagnosing ovarian cancer comprising steps (a), (b) and (c) of the third aspect.

As with the first and second aspects of the invention, step (a) of this third aspect of the invention may involve administering a plurality of nanoparticles to a subject to allow a biomolecule corona to form on the surface of said nanoparticles or incubating a plurality of nanoparticles in a biofluid sample taken from a subject to allow a biomolecule corona to form on the surface of said nanoparticles. Suitably, the subject is human. Suitably, the subject is a non-human animal such as a mouse, rat or monkey. Suitable routes of administration include but are not limited to intravenous, oral, intracerebral (including spinal), intraperitoneal and intra-occular. Conveniently, the route of administration is by intravenous injection. The biomolecule corona typically forms within less than 10 minutes from administration.

In an alternative embodiment, step (a) comprises incubating a plurality of nanoparticles in a biofluid sample taken from a subject to allow a biomolecule corona to form on the surface of said nanoparticles. Suitably, such incubation can be carried out ex vivo or in vitro (herein the term in vitro includes ex vivo). In this approach, the NP corona is formed in vitro by incubating the plurality of nanoparticles in a biofluid sample to be analyzed. Conveniently, this involves incubating at a suitable temperature, such as at about 37° C., for a suitable length of time. The biomolecule corona can form almost immediately, but typically the incubation is carried out for a period of 5-60 minutes, or more; such as 5, 10, 15, 20, 30, 40, 50, 60 or more minutes. Conveniently, the mixture can be subject to agitation, for example by way of an orbital shaker set at approximately 250 rpm to mimic in vivo conditions. Suitably, the biofluid sample from the subject to be analyzed has been previously taken and the sample extraction step is not part of the method.

In step (b) the nanoparticles and surface-bound biomolecule corona are isolated. Any isolation technique that is capable of preserving the surface-bound biomolecule corona is suitable. Conveniently, the nanoparticles with surface-bound biomolecule corona are isolated from the biofluid and purified to remove unbound Conveniently, the isolation is achieved by a method comprising size exclusion chromatography followed by ultrafiltration.

The method can be used to monitor disease progression, for example to monitor the efficacy of a therapeutic intervention. Suitably the disease is cancer. Suitably the method involves detecting tumour-specific cfNA over time. Suitably, the method involves detecting a nucleic acid target region.

According to a fourth aspect of the invention there is provided a method for monitoring disease progression in a subject, comprising:
(a) contacting a biofluid sample from the subject with a plurality of nanoparticles under conditions to allow a biomolecule corona to form on the surface of said nanoparticles;
(b) isolating the nanoparticles and surface-bound biomolecule corona; and
(c) analyzing the biomolecule corona for cfNA;
   wherein the degree of disease progression is determined based on the total cfNA level or cfNA level of a disease-specific biomarker relative to a reference amount.

In a particular embodiment, the disease is cancer.

In particular embodiments, the cancer is selected from the group consisting of: lung, ovarian, melanoma, prostate and blood cancer, including leukemia, lymphoma and myeloma. Suitably, the cancer is ovarian cancer.

In step (b) the nanoparticles and surface-bound biomolecule corona are isolated. Any isolation technique that is capable of preserving the surface-bound biomolecule corona is suitable. Conveniently, the nanoparticles with surface-bound biomolecule corona are isolated from the biofluid and purified to remove unbound biomolecules Conveniently, the isolation is achieved by a method comprising size exclusion chromatography followed by ultrafiltration.

In a particular embodiment, the reference amount is the amount detected at a previous time point. Suitably, the previous time point was at least 1 week, 2 weeks, 1 month, 3 months, 6 months, 12 months, 18 months, 24 months earlier.

In a particular embodiment, if the total amount of cfNA has increased compared to the reference amount it signifies that the patient's disease (e.g. cancer) has progressed and if the total amount of cfNA has decreased compared to the reference amount the patient's disease (e.g. cancer) has regressed.

In a particular embodiment of any aspect of the invention, the cfNA content of the biomolecule corona is quantitated using qPCR, such as real time qPCR.

The cfNA that forms or adsorbs onto the nanoparticles (either directly or indirectly by association with another biomolecules, such as a protein) can be subjected to genomic analysis by any technique of interest. Such analysis could be quantitating total nucleic acid, sequencing of the nucleic acid and/or undertaking one or more targeted genetic analyses using known molecular diagnostic techniques to test the genetic state of an individual, including assessing for genetic diseases; mendelian disorders; genetic mosaicism; predicting response to drug treatment; and/or diagnosing or monitoring a medical condition. In addition, the cfDNA-based disease diagnostics, in particular cancer diagnostics, contemplated herein possess the ability to detect a variety of genetic changes including somatic sequence variations that alter protein function, large-scale chromosomal rearrangements that create chimeric gene fusions, and copy number variation that includes loss or gain of gene copies.

Prior to the analysis of the cfNA in the surface-bound biomolecule corona it may be desirable to amplify the nucleic acid using the well-established technique of polymerase chain reaction (PCR). Alternatively, a nucleic acid library of the cell free nucleic acid in the surface-bound biomolecule corona could be generated.

A cfDNA library could be generated by the end-repair of isolated cfDNA, wherein fragmented cfDNA is processed by end-repair enzymes to generate end-repaired cfDNA with blunt ends, 5′-overhangs, or 3′-overhangs which can then be cloned into a suitable vector, e.g. plasmid, and used to generate a cfDNA clone library. Optionally, an adaptor is ligated to each end of an end-repaired cfDNA, and each adaptor comprises one or more PCR or sequencing primer binding sites. If desired, PCR can then amplify the initial cfDNA library. The amount of amplified product can be measured using methods known in the art, e.g., quantification on a Qubit 2.0 or Nanodrop instrument. In particular embodiments, a method for genetic analysis of cfDNA comprises: generating and amplifying a cfDNA library, determining the number of genome equivalents in the cfDNA library; and performing a quantitative genetic analysis of one or more target loci.

In particular embodiments, a method for genetic analysis of cfDNA comprises treating cfDNA with one or more end-repair enzymes to generate end-repaired cfDNA and ligating one or more adaptors to each end of the end-repaired cfDNA to generate a cfDNA library; amplifying the cfDNA library to generate cfDNA library clones; determining the number of genome equivalents of cfDNA library clones; and performing a quantitative genetic analysis of one or more target genetic loci in the cfDNA library clones.

The cfNA captured in the corona can be subjected to nucleotide sequencing by any method known in the art. DNA sequencing techniques include classic dideoxy sequencing reactions (Sanger method) using labelled terminators or primers and gel separation in slab or capillary, sequencing by synthesis using reversibly terminated labelled nucleotides or using allele specific hybridization to a library of labelled clones, Illumina/Solexa sequencing, pyrosequencing, 454 sequencing, and SOLiD sequencing. Separated molecules may be sequenced by sequential or single extension reactions using polymerases or ligases as well as by single or sequential differential hybridizations with libraries of probes.

An example of a suitable sequencing technique is Illumina sequencing which is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labelled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in various patent publications including: U.S. Pat. Nos. 7,960,120; 7,835,871; 7,232,656 and 6,210,891. With the advances in next generation sequencing, the cost of sequencing whole genomes has decreased dramatically, however the cost and time involved in sequencing entire genomes may not be practical or necessary. Instead, different genome partitioning techniques can be used to isolate smaller but highly specific regions of the genome for further analysis. Molecular Inversion Probe (MIP) technology, for instance, can be used to capture a small region of the genome for further examination, such as single nucleotide polymorphism (SNP) genotyping, allelic imbalance studies or copy number variation assessments (e.g. Hardenbol et al., “Highly multiplexed molecular inversion probe genotyping: over 10,000 targeted SNPs genotyped in a single tube assay”. Genome Res 15:269-75, 2005).

The present invention relates to a method of identifying a cell free nucleic acid biomarker in a biofluid.

The biofluid can be any fluid obtained or obtainable from a subject. The subject can be an animal. In a particular embodiment of any aspect of the invention the subject is a human. In particular embodiments, the subject is suffering from a disease (in a diseased state). Suitably, the subject is suffering from cancer.

In particular embodiments of any aspect of the invention, the biofluid is selected from blood, plasma, serum, saliva, sputum, urine, ascites, lacrimal, cerebrospinal and ocular fluids. In a particular embodiment, the biofluid is plasma.

Suitably the biofluid is a blood or blood fraction sample, such as serum or plasma.

The present invention involves the use of a plurality of nanoparticles. A plurality of nanoparticles can be a population of the same type of nanoparticle (a population of nanoparticles) or more than one population of nanoparticles, wherein each population is of a different type of nanoparticle; and so, when combined can be termed a heterogeneous population of nanoparticles (i.e. a plurality of distinct nanoparticle populations). Thus, in a particular embodiment the plurality of nanoparticles used is a heterogeneous population of nanoparticles.

In a particular embodiment, all the nanoparticles used in the method are of the same type of nanoparticle, and so can be termed a homogeneous population of nanoparticles.

The methods are applicable to any types of nanoparticles capable of attracting a biomolecule corona. In particular embodiments of any aspect of the invention, the nanoparticles are selected from liposomes, metallic nanoparticles (such as gold or silver), polymeric nanoparticles, fibre-shaped nanoparticles (such as carbon nanotubes) and two dimensional nanoparticles (such as graphene oxide nanoparticles). In a particular embodiment, the nanoparticles are PEGylated liposomes.

Conveniently, the nanoparticles are liposomes. Liposomes are generally spherical vesicles comprising at least one lipid bilayer. Liposomes are often composed of phospholipids. In a particular embodiment, the liposomes are composed of phospholipid molecules and functionalised amphiphilic molecules (eg. PEGylated DSPE). In a particular embodiment, the liposomes are composed of phospholipid molecules and functionalised amphiphilic molecules (eg. PEGylated DSPE) that are able to self-assemble into unilamellar vesicles. In a particular embodiment, the liposomes are PEGylated DSPE. Conveniently, the liposomes are able to encapsulate drug molecules in their inner aqueous phase.

The inventors have found that cfNA-containing coronas form on negatively charged nanoparticles. As nucleic acid is negatively charged this is surprising. In a particular embodiment, the nanoparticles are negatively charged. In a further embodiment, the nanoparticles are negatively charged liposomes.

The corona formed on the nanoparticles is a biomolecule corona. Conveniently, the biomolecule corona will typically comprise different classes of biomolecule, such as proteins, peptides, fatty acids, lipids, amino acids, amides, sugars and nucleic acids. Conveniently the biomolecule corona comprises cell free nucleic acid, such as cfDNA and/or cfRNA. Conveniently the biomolecule corona comprises one or more measurable biomarkers.

A biomarker, or biological marker, generally refers to a qualitative and/or quantitative measurable indicator of some biological state or condition. Biomarkers are typically molecules, biological species or biological events that can be used for the detection, diagnosis, prognosis and prediction of therapeutic response of diseases. Most biomarker research has been focused on measuring a concentration change in a known/suspected biomarker in a biological sample associated with a disease. Such biomarkers can exist at extremely low concentrations, for example in early stage cancer, and accurate determination of such low concentration biomarkers has remained a significant challenge.

The various aspects of the invention are directed to the detection/identification of one or more nucleic acid biomarkers. In a particular embodiment of any aspect of the invention, the biomarker(s) is/are cell-free nucleic acid, such as cfDNA or cfRNA. In a particular embodiment, the biomarker(s) is/are circulating tumour DNA (ctDNA).

Suitably, in any of the aspects of the invention, the cfNA is cell free ribonucleic acid (cfRNA) or cell free deoxyribonucleic acid (cfDNA). cfRNA can be any cell-free RNA including microRNA. cfDNA can be any cell free DNA, including genomic DNA. Suitably, the cfNA is fragmented. In a particular embodiment, the cfNA is nucleic acid released from a cancer cell. Such nucleic acid may comprise or house one or more mutations associated with the cancer. Such nucleic acid being assessed may be classed as a nucleic acid target region.

Fragmented cell-free DNA can be actively secreted into the bloodstream and is also passively released into circulation during cell death (apoptosis & necrosis) (Schwarzenbach et al. Nat. Rev. Cancer, 2011, 11, 426-437). In healthy individuals, cfDNA levels are usually extremely low, with elevated concentrations of cfDNA commonly triggered by pathological disease states, such a tumourigenesis, inflammation, ischemia, trauma and sepsis (Schwarzenbach et al. Nat. Rev. Cancer, 2011, 11, 426-437). Cell-free DNA is widely protected from nuclease digestion by its complexation with a core of histone proteins, known as nucleosomes (Snyder et al. Cell, 2016, 164, 57-68). Genomic analysis of the nucleic acid content in blood is of growing interest in recent years for diagnostic and disease monitoring applications, particularly with attempts to develop liquid biopsies for cancer from circulating tumour DNA (ctDNA) originating from tumour cells (Sorenson et al. Cancer Epidemiol. Biomarkers Prev., 1994, 3, 67-71).

Despite these preliminary indications of the interaction of nucleic acids with the surface of NPs, no studies have demonstrated formerly the presence of cfDNA in the biomolecule corona formed when NPs come into contact with human plasma.

In a particular embodiment of any aspect of the present invention, the amount or relative amount of total cfNA is determined.

In a particular embodiment of any aspect of the present invention, the amount or relative amount of total cfDNA is determined.

In a particular embodiment of any aspect of the present invention, the amount of cfNA in the corona is quantitated directly without prior cfNA extraction.

In a particular embodiment of any aspect of the present invention, the amount of cfDNA in the corona is quantitated directly without prior cfDNA extraction.

In some embodiments, it may be preferably to fragment the target nucleic acid. Nucleic acids, including genomic nucleic acids, can be fragmented using any of a variety of methods, such as mechanical fragmenting, chemical fragmenting, and enzymatic fragmenting. Methods of nucleic acid fragmentation are known in the art and include, but are not limited to, DNase digestion, sonication, mechanical shearing, and the like.

Genomic nucleic acids can be fragmented into uniform fragments or randomly fragmented. In certain aspects, nucleic acids are fragmented to form fragments having a fragment length and/or ranges of fragment lengths as required depending on the type of nucleic acid targets one seeks to capture and the design and type of probes such as molecular inversion probes (MIPs) that will be used. Chemical fragmentation of genomic nucleic acids can be achieved using methods such as a hydrolysis reaction or by altering temperature or pH. Nucleic acid may be fragmented by heating a nucleic acid immersed in a buffer system at a certain temperature for a certain period to time to initiate hydrolysis and thus fragment the nucleic acid. The pH of the buffer system, duration of heating, and temperature can be varied to achieve a desired fragmentation of the nucleic acid. Mechanical shearing of nucleic acids into fragments can be used e.g., by hydro-shearing, trituration through a needle, and sonication. Nucleic acid may also be fragmented enzymatically. Enzymatic fragmenting, also known as enzymatic cleavage, cuts nucleic acids into fragments using enzymes, such as endonucleases, exonucleases, ribozymes, and DNAzymes. Varying enzymatic fragmenting techniques are well-known in the art. In certain embodiments, the sample nucleic acid is captured or targeted using any suitable capture method or assay such as amplification with PCR, hybridization capture, or capture by probes such as MIPs.

In a particular embodiment of any aspect of the present invention, the isolated cfDNA is fragmented.

In a particular embodiment of any aspect of the present invention, the relative amount of the nucleic acid in the sample is determined by reference to a control nucleic acid in the sample. A control nucleic acid may be a nucleic acid sequence, such as a gene, that is representative of a wild-type/healthy level.

In a particular embodiment of any aspect of the present invention, a specific nucleic acid sequence within the cell-free nucleic acid is determined. Suitably, the specific nucleic acid is a nucleic acid target region. Suitably, the specific nucleic acid is indicative of a disease, such as being or comprising a disease-associated mutation. One example is the detection of activating mutations in epidermal growth factor receptor (EGFR) gene in certain patients with non-small cell lung cancer (NSCLC). Key activating mutations in EGFR include: a deletion in exon 19 (e.g. Del (746-750)) and the L858R point mutation that constitute approximately 90% of all EGFR activating mutations in NSCLC patients. The methods of the invention can be used to detect one or more EGFR activating mutations, or indeed, resistance mutations, and so can be used for diagnosis or monitoring purposes.

The methods of the invention can be used to monitor the effects of a therapeutic treatment. For example, a determination of the cfNA in a patient's biofluid can be conducted prior to a therapeutic intervention (such as chemotherapy, radiotherapy or administration of any therapeutic drug) and then at one or more time points during or after treatment. The change in cfNA detected can then be used to determine the effectiveness of the treatment.

Therefore, in some embodiments, the method may comprise an extra step, during or (preferably before step (a)), of administering a therapy to the subject, for example administering a drug molecule, such as for example, an anti-cancer compound. Suitable anti-cancer compounds include, but are not limited to, compounds with activity in cancers such as lung cancer, melanoma or ovarian cancer. In some embodiments, the anti-cancer compound is doxorubicin.

In a separate embodiment, there is provided a method for monitoring the changes in cfNA in a subject in response to therapy, comprising the step of a) contacting a plurality of nanoparticles with a biofluid from a therapeutically treated subject with cancer to allow a biomolecule corona to form on the surface of said nanoparticles.

In a particular embodiment of any aspect of the present invention, a change in total cfNA in a biofluid from a subject in response to therapy is monitored. In a particular embodiment of any aspect of the present invention, a change in cfNA of a tumour-associated genetic marker (e.g. mutation) in a biofluid from a subject in response to therapy is monitored. In a particular embodiment, the therapy comprises administration of a drug molecule to the subject. In a particular embodiment, the patient is being treated with an anti-cancer compound. Conveniently, the anti-cancer compound is doxorubicin.

In a particular embodiment, the invention relates to a method of identifying a nucleic acid biomarker from a biofluid, wherein the method comprises:
a. isolating a plurality of nanoparticles with surface-bound biomolecule corona from a biofluid sample taken from a subject in a diseased state; and
b. analyzing the biomolecule corona to identify the said nucleic acid biomarker.

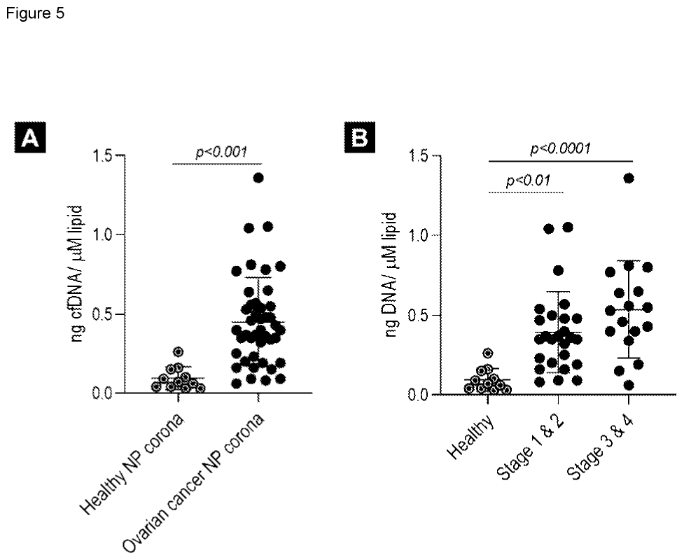

Surprisingly, the inventors have found that the total cfDNA biomolecule content of the biomolecule corona isolated after administering a plurality of nanoparticles to ovarian cancer subjects to allow a biomolecule corona to form on the surface of the nanoparticles is significantly higher in comparison to healthy subjects. FIG. 5 shows data to illustrate this surprising discovery. When normalised to post-purification liposome concentration, cfDNA was significantly higher in ovarian cancer samples (all stages, early stage (I and II) and late-stage (III and IV)) compared to healthy controls (p values=<0.001, <0.01 and <0.0001, respectively).

The cfNA content adsorbed onto the nanoparticle can therefore be used to detect or diagnose the disease state. cfNA detection in the NP corona can therefore be used to indicate the presence of disease in a subject.

In a particular embodiment of any aspect of the invention the cfNA is adsorbed onto the nanoparticle surface. Suitably, the cfNA is adsorbed onto the nanoparticle surface as part of a Nucleic Acid-protein complex. In particular embodiments, the Nucleic Acid-protein complex comprises one or more histone proteins, such as H2, H2B, H4, histone-lysine N-methyltransferase 2D and histone PARylation factor 1. In particular embodiments, the Nucleic Acid-protein complex is a DNA-protein complex.

The total biomolecule content of the cfNA biomolecule corona can be determined by any method capable of quantifying the level of said biomolecules in the surface-bound corona. In one embodiment, the biomolecule method involves determining the total nucleic acid content and this is suitably determined by qPCR. Total NA content can be gauged by measuring a reference gene, such as the RNase P gene (e.g. using The Applied Biosystems® TaqMan™ RNase P Detection Reagents Kit).

In one embodiment, the cfNA is detected directly from the NP corona. In another embodiment, the cfNA is purified from the corona before analysis. Purification of nucleic acid is well-known. A suitable kit for purifying circulating nucleic acid in a sample is QIAamp circulating nucleic acid extraction kit (QIAGEN).

Previously the inventors have also found that the total protein content determined by in vivo administration of the NP, followed by extraction and analysis is greater than if determined by incubating the plurality of nanoparticles ex vivo with a biofluid taken from the subject. It is expected that greater cfNA can be captured in the corona by the in vivo administration of the NP, followed by extraction and analysis method. The ex vivo method however also works efficiently and is likely to be attractive as it can be carried out on a sample previously taken from a subject and so is minimally invasive and avoids the need for the NP to be administered to the subject.

In a particular embodiment, the total cfNA content determined is at least between 1.2 and 5 fold higher than if determined by incubating the plurality of nanoparticles ex vivo with a biofluid isolated from the subject. Conveniently, total cfNA content determined is at least 1.5, 1.8, 2, 3, 4 or 5 fold higher than if determined by incubating the plurality of nanoparticles ex vivo with a biofluid isolated from the subject. Conveniently, the subject in this embodiment is a human or a non-human animal, such as a mouse, rat or monkey.

In the methods of the invention that involve administration of the nanoparticles to a subject, the route of administration of the nanoparticles may be by intravenous injection. The biomolecule corona typically forms within less than a few minutes from administration, so a biofluid sample comprising some of the introduced nanoparticles is then extracted from the subject; for example, by taking a blood sample, after a period of time to allow the corona to form. In particular embodiments, the biofluid sample comprising nanoparticles is extracted/removed from the subject at least 5 minutes after administration, such as at least 5, 6, 7, 8, 9, 10, 12, 15, 20, 30, 40, 60, 90, 120 minutes or more, after the nanoparticles were administered to the subject. The volume of the biofluid sample comprising nanoparticles extracted can be determined by the physician and will depend on the source of the biofluid sample. For example, if it is a blood sample, it may be in a volume of 2-20 ml. This method can be carried out with a human subject but suitably will be with a non-human subject.

When isolating the nanoparticles and surface-bound biomolecule corona, any isolation technique that is capable of preserving the surface-bound biomolecule corona is suitable. Conveniently, the nanoparticles with surface-bound biomolecule corona are isolated from the biofluid and purified to remove unbound and highly abundant biomolecules (for example albumin) to allow identification of lower abundant cfNA biomarkers. The method therefore allows minimization of any masking caused by the highly abundant proteins. Conveniently, the isolation is achieved by a method comprising size exclusion chromatography followed by ultrafiltration.

Thus, in particular embodiments of any aspect of the present invention, the nanoparticles with surface-bound biomolecule corona are isolated from the biofluid and purified to remove unbound and highly abundant biomolecules to allow identification of low abundant biomarkers. In particular embodiments, the nanoparticles with surface-bound biomolecule corona are isolated from the biofluid and purified to remove unbound and highly abundant biomolecules by a method comprising size exclusion chromatography followed by ultrafiltration.

As noted above, in addition to a determination of the total cfNA biomolecule content of the biomolecule corona, analysis of the biomolecule corona can also reveal qualitative and quantitative information regarding specific potential biomarkers. Such analysis can be carried out using any suitable techniques of capable of detecting said biomarkers. In a particular embodiment of the invention, the biomolecule corona is analysed by mass spectrometry, genomic sequencing or other technique for detecting nucleic acid. Conveniently, the biomolecule corona is analysed by mass spectrometry, which can allow qualitative and quantitative analysis of the biomolecule corona present on the nanoparticles. The cfNA content in the corona can be quantitated using real-time quantitative PCR (qPCR) assay. In a particular embodiment, the methods allow identification of unique biomolecules without the need for highly specialized and ultra-sensitive analytical mass spectrometry instrumentation such as using an UltiMate® 3000 Rapid Separation LC (RSLC, Dionex Corporation, Sunnyvale, CA) coupled to a LTQ Velos Pro (Thermo Fisher Scientific, Waltham, MA) mass spectrometer. Unique cfNA biomarkers can also be detected by nucleic acid sequencing, either direct on the corona or following polymerase chain reaction amplification of cfNA in the corona.

In a particular embodiment of the invention, the beneficial sensitivity and high level of precision provided by the method allows the identification of intracellular cfNA disease related biomarkers that are present in low abundance and would otherwise be very difficult to identify.

In a particular embodiment, the analysis is conducted on a single biofluid sample. Suitably, the sample is a plasma sample.

In addition to the identification of a single biomarker, the methods of the invention also provide the ability to identify panels of biomarkers (multiplexing). This approach can lead to increased sensitivity and specificity of detection. In a particular embodiment of any aspect of the invention, the biomarker is part of a panel of disease-specific biomolecule biomarkers. In a further embodiment, the panel comprises a combination of unknown and known disease-specific biomolecule biomarkers.

### EXAMPLES

### Materials and Methods

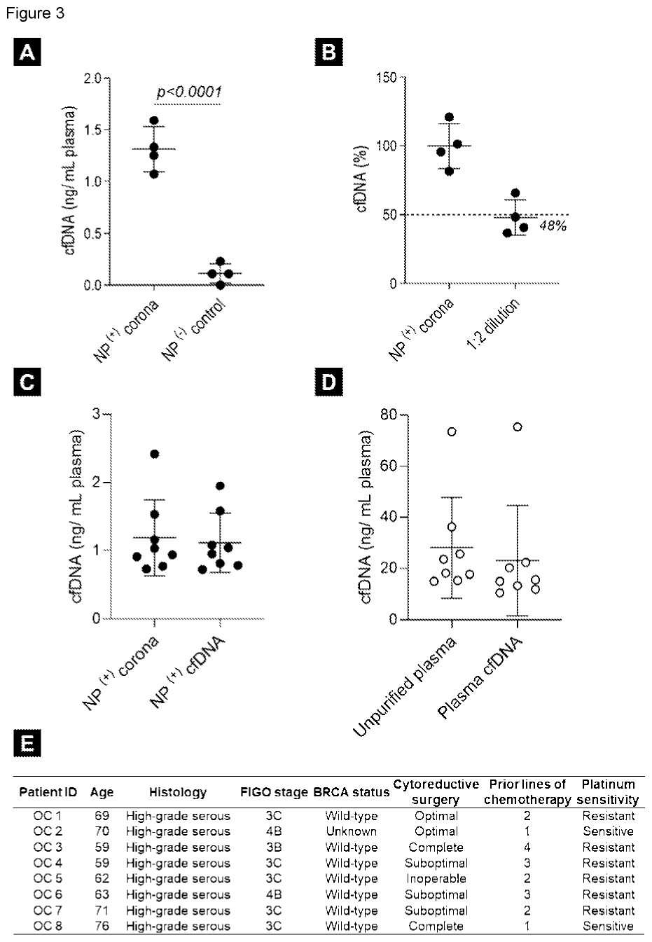

M1. Plasma samples. Healthy human female pooled K2EDTA plasma samples were purchased from BioIVT (West Sussex, UK) (Lot #HMN2528). All ovarian cancer K2EDTA plasma samples were collected by the MCRC Biobank (details provided in Table 1 and FIG. 3E). Individual age- and sex-matched K2EDTA plasma controls (female, 45-85 years old) were purchased from BioIVT (West Sussex, UK) (Table 1). All plasma samples were stored at −80° C.

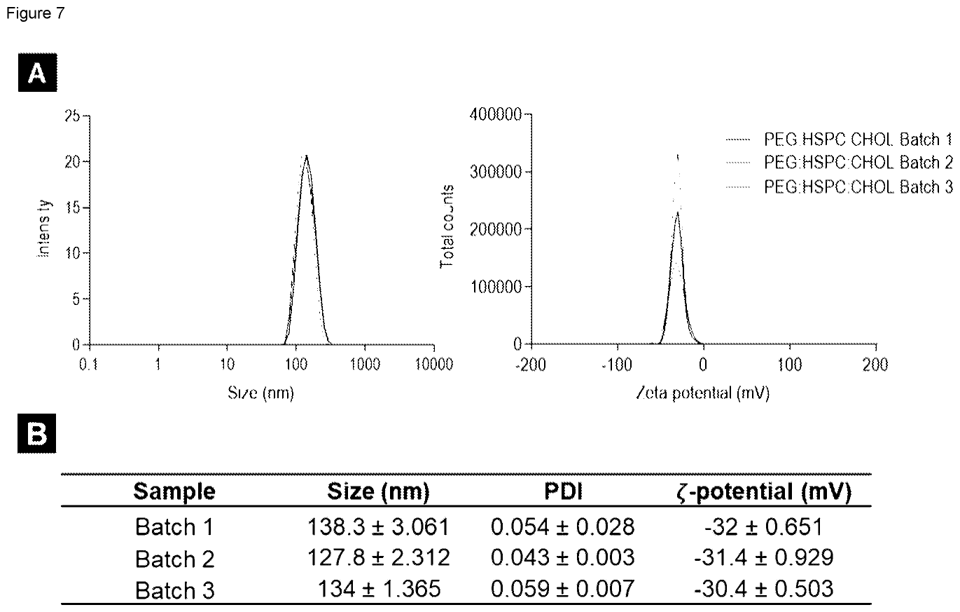

M2. Liposome preparation. HSPC:Chol:DSPE-PEG2000 (56.3:38.2:5.5) liposomes (Doxil® formulation) liposomes were prepared using the thin lipid film method followed by extrusion as described previously (Hadjidemetriou et al. ACS Nano, 2015, 9:8142-8156). All liposome batches were diluted to 12.5 mM, with the same batch of liposomes used for group comparisons. The physiochemical characteristics of the liposome batches are shown in FIG. 7.

M3. Dynamic light scattering (DLS) for size and zeta-potential measurements. Liposome size and surface charge were measured as described previously (Hadjidemetriou et al. ACS Nano, 2015, 9:8142-8156). Liposomes were diluted in distilled water and measured in size or capillary cuvettes using the Zetasizer Nano ZS (Malvern, Instruments, UK).

M4. Biomolecule corona formation (liposome plasma incubation and purification). Liposome and plasma incubations and purifications were performed as described previously (Hadjidemetriou et al. ACS Nano, 2015, 9:8142-8156). In brief, 820 μL human plasma and 180 μL PEGylated liposomes were incubated for 10 mins at 37° C., shaking at 250 rpm. Unbound proteins and other unknown biomolecules were removed by size exclusion chromatography (SEC) (Sepharose CL-4B columns (Sigma-Aldrich)) followed by membrane ultrafiltration (Vivaspin® columns (Sartorious, Fisher Scientific)). Samples were concentrated to 100 μL for characterisation or downstream processing. For characterisation of individual chromatographic fractions, samples were concentrated to 100 μL using 1,000,000 molecular weight cut off (MWCO) Vivaspin® membrane ultrafiltration columns (Sartorious, Fisher Scientific). Plasma controls were subjected to the same purification process for comparison.

M5. Circulating cell-free nucleic acid extraction. Cell-free nucleic acids were purified from ex vivo plasma samples, liposomal corona samples and plasma control samples using a QIAamp® Circulating Nucleic Acid Extraction kit and QIAvac 24 Plus vacuum manifold according to manufacturer's instructions (QIAGEN, Hilden, Germany). After an initial sample lysis step, cell-free nucleic acids were bound onto a silica-based purification column (QIAGEN mini column). Multiple washing steps were performed prior to elution of cell-free nucleic acids in buffer AVE (QIAGEN). All samples were eluted in a final volume of 50 μL.

M6. Cell-free DNA quantification. Cell-free DNA was measured using two real-time quantitative PCR (qPCR) assays. The single-copy RNase P probe real-time assay was performed using TaqMan® RNase P Detection Reagents kit (Life Technologies) and SensiFAST Probe Hi-ROX master mix (Bioline, Meridian Bioscience). All real-time qPCR reactions included 7.5 μL of 2× SensiFAST probe mastermix, 0.75 μL 20× RNase P primer/probe mix, 1.75 μL nuclease-free water (Ambion, Texas, USA) and 5 μL of sample. Cycling conditions included (95° C., 5 mins)×1, (95° C., 10 s; 60° C., 50 s)×40 and were performed on a LightCycler® 96 (Roche, Basel, Switzerland).

The multi-locus LINE-1 real-time qPCR assay was performed using primers described previously (Rago et al. Cancer Res., 2007, 67, 9364-9370), purchased from Integrated DNA Technologies (desalted, 25 nmol scale) using a robust Terra qPCR Direct SYBR Premix master mix (Takara Bio, USA). All real-time PCR reactions included 7.5 μL of 2× Terra qPCR Direct SYBR Premix master mix, 0.75 μL of each 10 μM forward and reverse primers), 5.75 μL nuclease-free water (Ambion, Texas, USA) and 1 μL of sample. Cycling conditions included (98° C., 2 mins)×1, (98° C., 10 s; 60° C., 15 s; 68° C., 30 s)×35 and were performed on a LightCycler® 96 (Roche, Basel, Switzerland).

Sample input was either corona-coated liposomes, purified cfDNA or plasma samples diluted 1:40. Plasma samples were only quantified using the LINE-1 real-time PCR assay in combination with the robust Terra qPCR Direct SYBR Premix master mix.

M7. Mass spectrometry. In-gel digestion of corona proteins was performed prior to LC-MS/MS analysis, as described previously (Hadjidemetriou et al. ACS Nano, 2015, 9:8142-8156). Digested proteins were analysed by LC-MS/MS using an UltiMate 3000 Rapid Separation LC (RSLC, Dionex Corporation, Sunnyvale, CA) plus Q Exactive Hybrid Quadrupole-Orbitrap (Thermo Fisher Scientific, Waltham, MA, USA) mass spectrometer system. Data were analysed using Mascot (Matrix Science UK) in combination with the SwissProt_2016_04 database (taxonomy human). Progenisis QI software (version 4.3.2, Proteome Software Inc.) was used for relative protein quantification based on spectral counting and statistical analyses (One-way analyses of variance (ANOVA)).

M8. Statistical analysis. Statistical comparisons of these data were performed using GraphPad Prism v.8.2.0. For comparisons of three groups or more, one-way ANOVA tests were performed followed by the Tukey's multiple comparison test (adjusted p values<0.05 were considered significant). For comparisons of two groups unpaired student t-tests were performed (FDR-adjusted p values<0.05 were considered significant). All data averages were presented as mean±standard deviation (SD).

M9. Ethical Approvals: This project has research ethics approval under the Manchester Cancer Research Centre (MCRC) Biobank Research Tissue Bank Ethics (NHS NW Research Ethics Committee 18/NW/0092). All participants provided written informed consent to participate in this study.

### Example 1

### 1.1 Plasma Incubation and Biomolecule Corona Formation

To evaluate the cfDNA content of the biomolecule corona, human plasma samples obtained from healthy volunteers were incubated (37° C., 10 minutes, 250 rpm) with PEGylated liposomes (HSPC:Chol:DSPE-PEG2000), a formulation which constitutes the basis of the anti-cancer agent Doxil® (FIG. 7). Liposomes were employed in this study due to their extensive protein corona characterisation, their use in nucleic acid-based biotechnology applications and more recently due to their promise as a proteomic enrichment tool (e.g. see Hadjidemetriou et al. Adv. Mater., 2019, 31, 1-9; Rasoulianboroujeni et al. Mater. Sci. Eng. C, 2017, 75, 191-197).

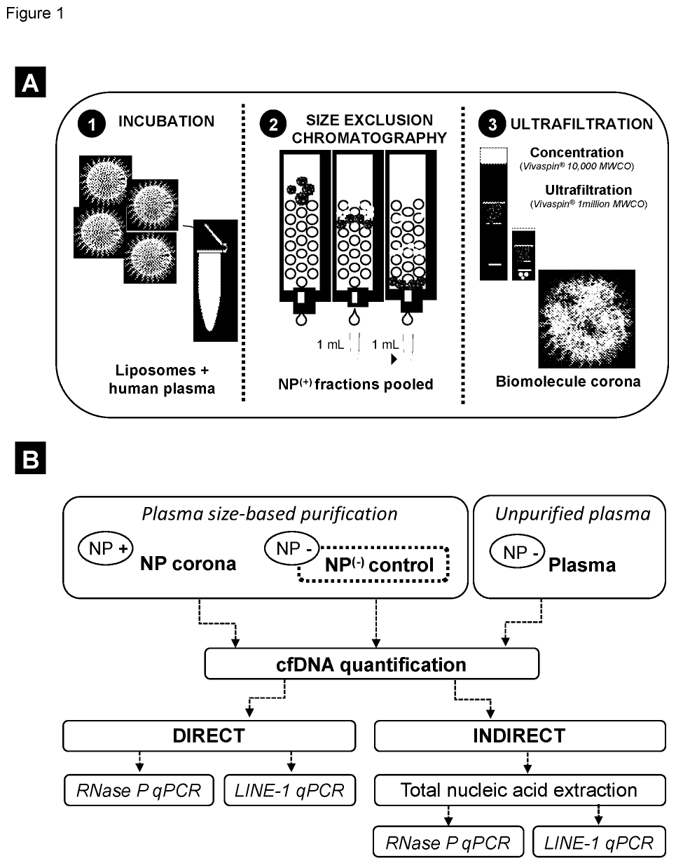

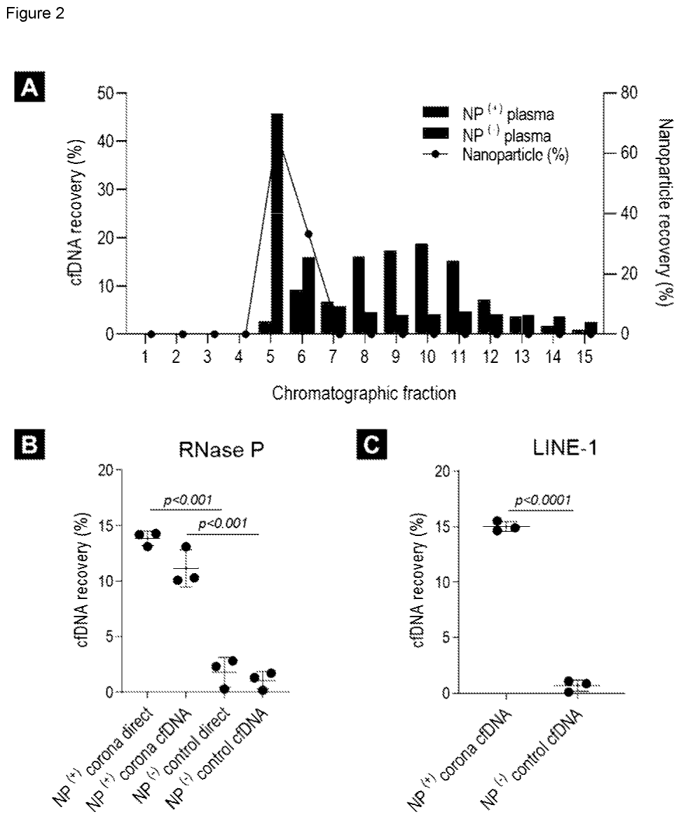

In order to assess the potential interaction of cfDNA with PEGylated liposomal surfaces, plasma-incubated liposomes were purified by size exclusion chromatography (SEC); represented in FIG. 1A), as described previously (Hadjidemetriou et al. ACS Nano, 2015, 9:8142-8156). Plasma control samples (without prior incubation with liposomes) were subjected to the exact same purification process. SEC column-eluted cfDNA was extracted from chromatographic fractions 1-15, using a QIAamp® circulating nucleic acid extraction kit (QIAGEN) and subsequently quantified using robust and highly sensitive LINE-1 real-time qPCR assay (FIG. 2A). Stewart assay was also performed in order to quantify the amount of liposomes eluted.

As illustrated in FIG. 2A and in agreement with our previous studies (Hadjidemetriou et al. ACS Nano, 2015, 9:8142-8156), corona-coated liposomes were eluted in chromatographic fractions 5 and 6, while no detectable lipid content was found in the fractionated plasma control. Distribution of cfDNA across chromatographic fractions 1-15 revealed significant differences between plasma-incubated liposomes and the matched plasma control. In the case of the plasma-incubated liposome sample the majority of cfDNA (45.8%) was eluted in chromatographic fraction 5, which also contained the largest population of liposome NPs (66.7%), while liposome-free fractions 7-15 contained relatively small quantities of cfDNA (<6%). In contrast, a normal distribution of cfDNA was evident in the fractionated plasma control, with the highest amount of cfDNA detected in fraction 10 (18.8%). Notably, in the absence of NPs, only 2.6% of the cfDNA content was detected in fraction 5. The striking difference in cfDNA distribution between corona-coated liposomes and the fractionated plasma control suggests that a significant proportion of cfDNA eluted in fraction 5 could be associated with the eluted liposomes.

Our data provide the first experimental evidence of the presence of cfDNA in the NP corona samples and show that the majority of cfDNA detected is associated with the surface of liposomes and is not passively co-eluted during purification (FIGS. 2A-C).

### 1.2 Quantitative Detection of cfDNA in the Liposome Corona

To further purify corona-coated liposomes from any remaining protein complexes and/or unbound cfDNA, chromatographic fractions 5 and 6 were pooled, concentrated and subsequently washed three times using a membrane ultrafiltration column (Vivaspin®, 1 million MWCO) (Hadjidemetriou et al. Biomaterials, 2019, 188, 118-129; M. Hadjidemetriou et al. Adv. Mater., 2019, 31, 1-9; and, Al-Ahmady et al. J. Control. Release, 2018, 276, 157-167).

To determine the total cfDNA content of the liposomal corona two different real-time qPCR assays were utilised, as outlined in FIG. 1B. A real-time qPCR approach was chosen as the concentration of cfDNA in blood commonly falls below the lower limit of detection for absorbance and fluorescence-based DNA quantification methods. Initially, a standardised TagMan® RNase P detection real-time qPCR assay (Applied Biosystems®) was used to quantify the cfDNA content of the biomolecule corona in healthy plasma samples. As illustrated in FIG. 2B, the concentration of cfDNA measured in the corona samples was significantly higher in comparison to plasma control samples that underwent the full purification process (adjusted p-value<0.0001). A small amount of cfDNA was identified in purified plasma controls, suggesting a co-elution of a small population of cfDNA molecules complexed with large proteins or within extracellular vesicles (FIG. 2B). These data suggested that most of the cfDNA quantified in corona samples is associated (directly or indirectly) with the surface of liposomes and was not passively co-eluted in a size-dependent manner.

In order to investigate whether the presence of proteins and/or other molecules in the biomolecule corona affects the direct quantification of cfDNA, we compared the amount of cfDNA with and without prior extraction (QIAGEN's QIAamp® circulating nucleic acid extraction kit). Comparable amounts of cfDNA were detected using the TaqMan® RNase P assay both in corona-coated liposome samples and in cfDNA subsequently purified from the same corona samples (FIG. 2B). These data indicated that the real-time qPCR assay was not significantly inhibited by other molecules present in the corona, allowing direct cfDNA measurements in the presence of lipid-based NPs and complex biofluid contaminants. To further investigate qPCR inhibition in NP-corona samples, a 2-fold dilution was performed prior to real-time qPCR quantification (FIGS. 3A&B). The cfDNA quantity of the 1:2 diluted corona sample was approximately half that of the original measurement (48%), providing further evidence to support the lack of RNase P qPCR inhibition in these direct real-time PCR measurements. The concentration of cfDNA in the NP-corona samples and plasma controls (with no NPs) was confirmed with a robust and sensitive LINE-1 qPCR assay (FIG. 2C). Both assays produced similar values, with RNase P and LINE-1 quantification methods consistently detecting significantly more cfDNA in corona samples when compared to plasma controls, as shown in FIG. 2C.

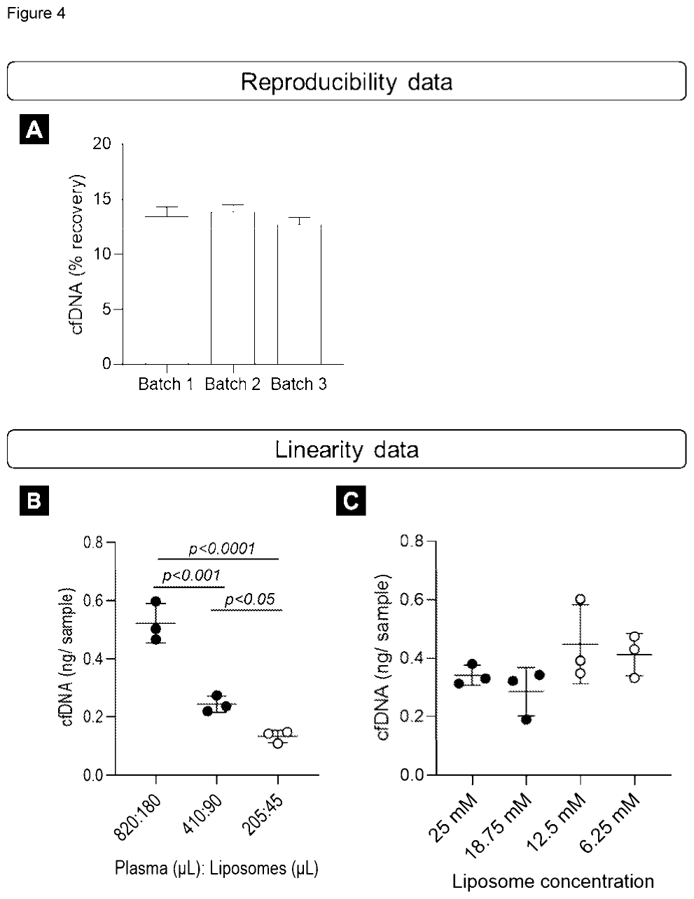

In terms of reproducibility, the percentage of cfDNA recovered with liposomal NPs was consistent across healthy plasma and liposome batches (FIG. 4A). In addition, plasma linearity experiments revealed a significant reduction in total cfDNA content when plasma input volume was lowered, while the plasma:NP ratio was maintained (adjusted p-values<0.01 for both 410 μL & 205 μL of plasma when compared to 810 μL) (FIG. 4B). In contrast to the linear relationship observed between plasma volume and cfDNA concentration, altering the concentration of liposome NPs did not significantly affect the amount cfDNA recovered (FIG. 3C). Combined, these data suggested that at the NP concentrations investigated, liposomes interacted reproducibly with a sub-population of plasma cfDNA molecules and that a NP:plasma [μL:μL] ratio of 0.2 was found optimal to recover this fraction of cfDNA.

Direct quantification of cfDNA was possible within complex lipid-based biomolecule corona samples without prior cfDNA extraction using the QIAamp circulating nucleic acid extraction kit (QIAGEN). In addition, cfDNA was successfully purified from lipid NPs using a standard cfDNA extraction kit, highlighting the compatibility of lipid-based NPs with downstream purification and quantification methods.

### 1.3 Detection of cfDNA in Ovarian Carcinoma Liposomal Corona Samples

To establish whether cfDNA could also be detected on the surface of liposomes incubated ex vivo with plasma obtained from cancer patients, corona-coated liposomes were prepared upon incubation and purification from plasma samples obtained from 43 patients with ovarian cancer (18 patients with FIGO stage I, 8 with stage II, 12 with stage III and 5 with stage IV) (Table 1).

TABLE 1
Table outlining clinical characteristics of ovarian cancer patient cohort and healthy normal
volunteers (HNVs). Details include sample number (n), age-range (years), histological subtype,
germline BRCA mutation status, baseline CA125 concentration (U/mL), prior lines of chemotherapy
and platinum sensitivity.

| | Ovarian cancer patients | | | | | | | | |
|---|---|---|---|---|---|---|---|---|---|
| | Healthy | Stage 1 | | Stage 2 | | Stage 3 | | Stage 4 | |
| Sample number | 11 | 18 | | 8 | | 12 | | 5 | |
| Age-range (median) | 40-59 (51) | 21-87 | (59) | 32-77 | (60) | 37-74 | (62) | 36-67 | (48) |
| Histological subtype | N/A | Mucinous-11 | (61%) | Serous-6 | (75%) | Serous-9 | (75%) | Serous-5 | (100%) |
| | | Serous-5 | (28%) | Endometroid-2 | (25%) | Adenocarcinoma | (17%) | | |
| | | Clear cell-1 | (5.5%) | | | (NOS)-2 | | | |
| | | Endometroid 1 | (5.5%) | | | Carcinosarcoma-1 | (8%) | | |
| Germline BRCA | N/A | Positive-0 | (0%) | Positive-0 | (0%) | Positive-1 | (8%) | Positive-1 | (20%) |
| status | | Negative-1 | (5.5%) | Negative-3 | (37.5%) | Negative-0 | (0%) | Negative-3 | (60%) |
| | | Unknown-17 | (94.5%) | Unknown-5 | (62.5%) | Unknown-11 | (92%) | Unknown-1 | (20%) |
| Baseline CA125 (U/mL) | N/A | Median 60 | (12-550) | Median 29.5 | (4-600) | Median 16 | (7-358) | Median 15 | (9-396) |
| Prior lines of | N/A | 0 | (94%) | 0 | (62.5%) | 0 | (50%) | 0 | (20%) |
| chemotherapy | | 2 | (6%) | 1 | (37.5%) | 1 | (42%) | 1 | (80%) |
| | | | | | | 2 | (8%) | | |
| Platinum sensitivity | N/A | Sensitive-6 | (33%) | Sensitive-3 | (37.5%) | Sensitive- 1 | (8%) | Sensitive-2 | (40%) |
| | | Resistant-1 | (6%) | Resistant-1 | (12.5%) | Resistant- 0 | (0%) | Resistant-1 | (20%) |
| | | Unknown-11 | (61%) | Unknown-4 | (50%) | Unknown-11 | (92%) | Unknown-2 | (40%) |

Patients with ovarian cancer classified across all stages of the disease were included in the study to determine whether cfDNA could be detected in NP corona samples both at early stages and as the disease progressed. These samples were quantified directly using a robust high sensitivity LINE-1 qPCR assay and compared to corona samples from 11 healthy aged matched females (FIG. 5). When normalised to post-purification liposome concentration, cfDNA was significantly higher in ovarian cancer samples (all stages, early stage (I and II) and late-stage (III and IV)) compared to healthy controls (p values=<0.001, <0.01 and <0.0001, respectively) (FIG. 5). In addition, average cfDNA content increased from early (FIGO stage I and II) to late stage (FIGO stage III and IV), although this was not statistically significant (FIG. 5B). These data are consistent with previous studies that have proposed quantification cfDNA as a diagnostic and prognostic biomarker for ovarian cancer, with increased cfDNA levels detected with disease progression (No et al., Anticancer Res., 2012, 32, 3467-71; Kamat et al., Cancer, 2010, 116, 1918-1925).

To determine whether direct cfDNA quantification in ovarian cancer corona samples would be inaccurate and skewed, with real-time qPCR inhibition increasing disproportionately with cancer stage, we compared cfDNA concentration in purified and unpurified samples for eight late-stage (stage III n=6, stage IV n=2) high-grade serous ovarian cancer samples (details provided in FIG. 3E). Similar cfDNA concentrations were measured for both unpurified ovarian cancer corona samples and their respective purified cfDNA samples (FIG. 3C). This suggests that real-time qPCR was not significantly inhibited in these biomolecule corona qPCR reactions and that no significant cfDNA loss occurred during cfDNA extraction using QIAGEN's QIAamp® circulating nucleic acid extraction kit. We were also able to measure the cfDNA content directly in ovarian cancer plasma samples (diluted 1:40), which again showed no significant difference from the respective purified plasma cfDNA samples (FIG. 3D).

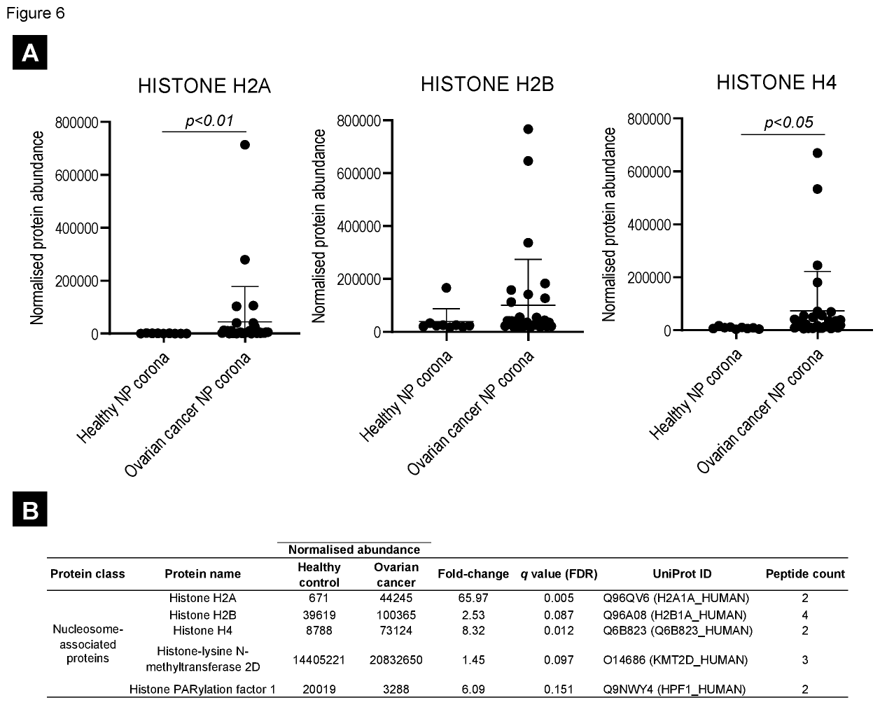

Mass spectrometry (LC-MS/MS) proteomic analysis was then performed on the 43 samples from ovarian cancer patients and the 11 samples from healthy controls to investigate whether proteins known to associate with cfDNA could be detected in the biomolecule corona (FIG. 6). Histone proteins, H2A, H2B and H4, which are found within the core nucleosome complex, were detected in the biomolecule corona and were identified at significantly higher levels in ovarian cancer samples relative to healthy controls (FIG. 6A). Two additional nucleosome-interacting proteins were identified in these samples, namely histone-lysine N-methyltransferase 2D and histone PARylation factor 1 (FIG. 6B) (Kowal et al., Proc. Natl. Acad. Sci. U.S.A., 2016, 113, E968-E977) Combined, these data confirmed the presence of cfDNA in the biomolecule corona of liposomes and suggested an indirect interaction which is potentially mediated via the nucleosome complex.

### 1.4

The PEGylated liposomes used in this study have a negative surface charge (FIG. 7A), therefore it was considered unlikely that DNA molecules would be bound directly onto the liposome surface via electrostatic interactions. Considering that cfDNA is protected within nucleosome complexes in the blood (Snyder et al., Cell, 2016, 164, 57-68), we hypothesised that cfDNA may not be directly bound onto the liposome surface, but through the adsorption of DNA-protein complexes. This indirect mechanism of adsorption was further supported by the identification of positively charged nucleosome core proteins, including histone proteins H2A, H2B and H4, in the biomolecule corona by LC-MS/MS analysis (FIG. 6). Of note, our group has previously detected histone proteins in human ex vivo, human in vivo and mouse in vivo liposomal corona samples (Hadjidemetriou et al., Biomaterials, 2019, 188, 118-129; Papafilippou et al., Nanoscale, 2020, 12, 10240-10253; and Hadjidemetriou et al. Nano Today, 2020, 34, 100901). Moreover, human histone proteins (H2B and H4) have also been identified in the healthy corona of colloidal gold NPs (Dobrovolskaia et al., Nanomedicine Nanotechnology, Biol. Med., 2014, 10, 1453-1463). Furthermore, De Paoli and colleagues demonstrated that calf thymus histone H1 binds to carboxylated-multiwalled carbon nanotubes (CNTCOOH) (De Paoli et al., Biomaterials, 2014, 35, 6182-6194). In addition, consistent cfDNA recovery across batches (FIG. 4A) suggested its reproducible and stable interaction with the liposomal surface as part of the biomolecule corona.

### 1.5 Discussion

Our data demonstrated that the corona-containing cfDNA levels were significantly higher in the biomolecule coronas formed upon incubation with plasma samples obtained from ovarian cancer patients (both early- and late-stages) in comparison to healthy controls (FIG. 5). It has been widely reported that total cfDNA is elevated in many different cancer types, such as colorectal, glioblastoma, colorectal and breast cancer, and increases with progression of the disease (Kamat et al., Cancer, 2010, 116, 1918-1925; Valpione et al., Eur. J. Cancer, 2018, 88, 1-9; Bagley et al., Clin. Cancer Res., 2020, 26, 397-407; Hao et al., Br. J. Cancer, 2014, 111, 1482-1489; Fernandez-Garcia et al., Breast Cancer Res., 2019, 21, 149). It is important to clarify that DNA originating from the tumour frequently only makes up a small proportion of total cfDNA, with the majority of DNA molecules released from non-malignant cells (Snyder et al. Cell, 2016, 164, 57-68; Mizuno et al., Sci. Rep., 2019, 9, 1-11). Moreover, healthy cfDNA detected in individuals with cancer is commonly of heamatopoietic origin and can be attributed to increased white blood cell turnover and chemotherapeutic- and/or radiation-induced cell death (Snyder et al. Cell, 2016, 164, 57-68; Valpione et al. Eur. J. Cancer, 2018, 88, 1-9). The elevated cfDNA detected in ovarian cancer patients in this study may therefore be attributable to cfDNA released from normal cells.

The ability to conduct genomic analysis on NP-corona offers up the ability to discover and analyse cancer-specific biomarkers in the NP corona. This approach could offer significant advantages over current purification methods, which lack the sensitivity required to detect ctDNA in small volumes of human plasma in patients with low tumour burden, especially pertinent to the challenge of early cancer detection.

Previous observations have shown that physiological diseased states affects blood composition, which is reflected in corona formation (Hadjidemetriou et al., Biomaterials, 2019, 188, 118-129). For example, our group has previously shown that protein corona quantitatively and qualitatively changed in the presence of tumorigenesis, with higher total amount of protein found to interact with intravenously injected liposomes recovered from melanoma and lung adenocarcinoma tumour-bearing mice in comparison to healthy controls (Hadjidemetriou et al., Biomaterials, 2019, 188, 118-129). Further analysis revealed that histone H2A was significantly upregulated in the in vivo lung adenocarcinoma corona samples (Hadjidemetriou et al., Biomaterials, 2019, 188, 118-129). Therefore, the increased amount of nucleosome-related proteins in ovarian cancer samples is likely to extend to other cancer types and NP classes, as a general reflection of increased cfDNA and histone content, commonly seen in cancer (Schwarzenbach et al. Nat. Rev. Cancer, 2011, 11, 426-437; Kuroi et al., Int. J. Oncol., 2001, 19, 143-148; Holdenrieder et al., in Annals of the New York Academy of Sciences, New York Academy of Sciences, 2008, vol. 1137, pp. 180-189; Kumar et al., Clin. Lung Cancer, 2010, 11, 36-44). In terms of other pathological conditions, our previous analysis of the ex vivo corona formed in the plasma of sepsis patients revealed a significant increase in histone H2B compared to plasma from both systemic inflammatory response syndrome (SIRS) patients and healthy controls (Papafilippou et al., Nanoscale, 2020, 12, 10240-10253). Comprehensive comparison of ‘healthy’ and ‘diseased’ protein coronas has been found to be a very promising enrichment tool for plasma analysis, enabling proteomic discovery of low abundant, diagnostic biomarkers (Hadjidemetriou et al. Adv. Mater., 2019, 31, 1-9; Papafilippou et al., Nanoscale, 2020, 12, 10240-10253).

In recent years, other cell-free nucleic acids, such as miRNAs, have received growing interest as disease biomarkers (de Miguel Pérez et al. Sci. Rep., 2020, 10, 1-13) and although extensive characterisation of the NP corona nucleic acid content was beyond the scope of this study, it remains an important avenue of future research. In addition, epigenetic analysis of ctDNA, such as differential methylation profiles can also provide cancer-specific signatures (Liu et al., Ann. Oncol., 2020, 31, 745-759). Intriguingly, methyl-cytosines have been shown to display a strong affinity to bare metal surfaces, including gold nanoparticles (Sina et al., Nat. Commun., 2018, 9, 1-13; Nguyen and Sim, Biosens. Bioelectron., 2015, 67, 443-449). Furthermore, post translational modifications of histone proteins have also been widely associated with tumourigenesis and have been previously detected in the plasma of cancer patients (Esteller, Nat. Rev. Genet., 2007, 8, 286-298; Kurdistani, Br. J. Cancer, 2007, 97, 1-5; Fraga and Esteller, Cell Cycle, 2005, 4, 1377-1381; Deligezer et al., Clin. Chem., 2008, 54, 1125-1131; Gezer et al., Oncol. Lett., 2012, 3, 1095-1098; Gezer et al., Int. J. Mol. Sci., 2015, 16, 29654-29662). The molecular complexes of cell-free nucleic acids contained with the biomolecule corona need to be fully elucidated in order to establish the scope for a sensitive blood-based biomarker enrichment tool.

The molecular information contained within the NP corona is far richer than originally described and has been shown to contain a diverse array of biomolecules including proteins, lipids, metabolites and now cfDNA. This complex coating on the surface of NPs has the potential to be able to enhance nano-drug delivery and NP uptake, but perhaps most significantly, offers the potential to provide greater sensitivity for liquid biopsies.

This study has shown that cell-free DNA is present in the biomolecule corona that forms around lipid-based NPs, upon incubation with human plasma. The cfDNA content of the biomolecule corona could be directly quantified in the presence other biomolecules (e.g. proteins) using conventional real-time qPCR assays. Furthermore, proteomic analysis of the biomolecule corona by LC-MS/MS revealed the presence of nucleosome complex proteins, suggesting an indirect protein-mediated interaction of cfDNA with NPs. Notably, the amount of cfDNA was found to be significantly higher in the coronas formed in early- and late-stage cancer patient plasma samples compared to healthy controls, indicating a disease-specific biomolecule corona formation. This study highlights the potential exploitation of the biomolecule corona as a novel blood-analysis nanoscale tool and that multi-omic analysis can be carried out on the NP-corona, such as from a single sample, either sequentially or in parallel.

1. A method of identifying cell free nucleic acid (cfNA) in a biofluid, wherein the method comprises:
(a) contacting a plurality of nanoparticles with a biofluid of a subject to allow a biomolecule corona to form on the surface of said nanoparticles;
(b) isolating the nanoparticles and surface-bound biomolecule corona; and
(c) analyzing the biomolecule corona for cfNA.

2. The method according to claim 1, wherein step (a) is performed in vivo by administering a plurality of nanoparticles to a subject or in vitro using a biofluid sample that has been taken from a subject.

3. The method according to claim 1, wherein the nanoparticles are administered to a subject by intravenous injection.

4. The method according to claim 1, wherein the plurality of nanoparticles are incubated in the test biofluid sample in vitro under conditions to allow a biomolecule corona to form on the surface of said nanoparticles.

5. The method according to claim 1, wherein the analysis is conducted on a single biofluid sample; optionally wherein the sample is selected from the group consisting of: blood, plasma, serum, saliva, sputum, urine, ascites, lacrimal, cerebrospinal and ocular fluids; optionally wherein the sample is a blood or blood fraction sample, such as serum or plasma.

6. (canceled)

7. (canceled)

8. The method according to claim 1, wherein the nanoparticles are selected from liposomes, metallic nanoparticles (such as gold or silver), polymeric nanoparticles, fibre-shaped nanoparticles (such as carbon nanotubes and two dimensional nanoparticles such as graphene oxide nanoparticles); optionally wherein the nanoparticles are PEGylated liposomes.

9. (canceled)

10. The method according to claim 8, wherein the nanoparticles are negatively charged.

11. The method according to claim 1, wherein the cfNA is cell-free DNA.

12. The method according to claim 1, wherein the nanoparticles with surface-bound biomolecule corona are isolated from the biofluid and purified to remove unbound and highly abundant biomolecules to allow identification of low abundant biomarkers; optionally wherein the nanoparticles with surface-bound biomolecule corona are isolated from the biofluid and purified to remove unbound and highly abundant biomolecules by a method comprising size exclusion chromatography followed by ultrafiltration.

13. (canceled)

14. The method according to claim 1, wherein the biofluid sample analyzed is from a subject in a diseased state, such as cancer like one selected from the group consisting of: ovarian, lung, prostate, melanoma and blood cancer, including leukemia, lymphoma and myeloma.

15. The method according to claim 1, wherein the amount or relative amount of total cell-free nucleic acid (cfNA) is determined.

16. The method according to claim 1, wherein the amount of cfNA in the corona is quantitated directly without prior cfNA extraction.

17. The method according to claim 15, wherein the relative amount of the nucleic acid in the sample is determined by reference to a control nucleic acid in the sample.

18. The method according to claim 1, wherein a specific nucleic acid sequence within the cell-free nucleic acid is determined

19. The method according to claim 18, wherein the specific nucleic acid is indicative of a disease, such as being or comprising a disease-associated mutation.

20. The method according to claim 1, wherein a change in total cfNA in a biofluid from a subject in response to therapy is monitored, optionally wherein the therapy comprises administration of a drug molecule to the subject, optionally wherein the drug molecule is an anti-cancer compound.

21. A method for detecting a disease state in a subject comprising:
(a) contacting a biofluid sample from the subject with a plurality of nanoparticles under conditions to allow a biomolecule corona to form on the surface of said nanoparticles;
(b) isolating the nanoparticles and surface-bound biomolecule corona; and
(c) analyzing the biomolecule corona for a disease-specific cfNA biomarker, which is determinative of the presence of a disease in said subject.

22. A method for diagnosing cancer in a subject, comprising:
(a) contacting a biofluid sample from the subject with a plurality of nanoparticles under conditions to allow a biomolecule corona to form on the surface of said nanoparticles;
(b) isolating the nanoparticles and surface-bound biomolecule corona; and
(c) analyzing the biomolecule corona for cfNA;
wherein an increase in total cfNA level relative to a reference or control amount is indicative of the presence of cancer.

23. A method for monitoring disease progression in a subject, comprising:
(a) contacting a biofluid sample from the subject with a plurality of nanoparticles under conditions to allow a biomolecule corona to form on the surface of said nanoparticles;
(b) isolating the nanoparticles and surface-bound biomolecule corona; and
(c) analyzing the biomolecule corona for cfNA;
wherein the degree of disease progression is determined based on the total cfNA level or cfNA level of a disease-specific biomarker relative to a reference amount,
optionally wherein the reference amount is the amount detected at a previous time point.

24. The method according to claim 23, wherein the disease is cancer and if the total amount of cfNA has increased compared to the reference amount the cancer in the patient's has progressed and if the total amount of cfNA has decreased compared to the reference amount the patient's cancer has regressed.
